(19) United States
(12) Patent Application Publication
(10) Pub. No.: US 2026/0048180 A1
(43) Pub. Date: Feb. 19, 2026

(54) Citrate-Based Interpositional Patch System for Rotator Cuff Repair

(71) Applicant: Acuitive Technologies, Inc., Allendale, NJ (US)

(72) Inventors: Minh-Tuan Richard Tran, Ho-Ho-Kus, NJ (US); Michael P. McCarthy, Ho-Ho-Kus, NJ (US); Rui J. Ferreira, Livingston, NJ (US); Andrew House, Elmwood Park, NJ (US)

(73) Assignee: Acuitive Technologies, Inc., Allendale, NJ (US)

(21) Appl. No.: 19/302,853

(22) Filed: Aug. 18, 2025

Related U.S. Application Data
(60) Provisional application No. 63/684,598, filed on Aug. 19, 2024.

(51) Int. Cl. A61L 27/46; A61L 27/44; A61L 27/56; A61L 27/58

(52) U.S. Cl. CPC A61L 27/46; A61L 27/446; A61L 27/56; A61L 27/58; A61L 2430/24

(57) ABSTRACT

A synthetic rotator cuff repair scaffold/patch system fabricated from citrate-based materials is provided. The biodegradable scaffold/patch system includes (i) a citrate component and/or other carboxylic acid components, (ii) a polyol, and (iii) particulate inorganic material. The patch system includes a patch that defines a patch thickness and may define a porosity gradient across the patch thickness.

### CROSS-REFERENCE TO RELATED APPLICATION

The present application claims priority benefit to a US provisional patent application entitled “Citrate-Based Interpositional Patch System for Rotator Cuff Repair,” which was filed on Aug. 19, 2024, and assigned Ser. No. 63/684,598. The entire content of the foregoing provisional patent application is incorporated herein by reference.

### BACKGROUND

### Technical Field

The present disclosure is directed to citrate-based polymer-bioceramic compositions that have beneficial utility as interpositional patches for rotator cuff repair.

### Background Art

Rotator cuff disease is a prevalent condition affecting up to 22.4% of adults over 40. [See, Kuhn, J. E. (2023). Prevalence, Natural History, and Nonoperative Treatment of Rotator Cuff Disease. Operative Techniques in Sports Medicine, 31(1), 150978-150978. https://doi.org/10.1016/j.otsm.2023.150978] Partial or full rotator cuff tears cause patients to experience pain and limited mobility that can progressively worsen. For example, 80% of partial-thickness rotator cuff tears become full-thickness tears within 2 years if medical intervention is not sought. [See, Thangarajah et al., (2022). Optimal Management of Partial Thickness Rotator Cuff Tears: Clinical Considerations and Practical Management. Orthopedic Research and Reviews, Volume 14, 59-70. https://doi.org/10.2147/orr.s348726] Furthermore, the re-tear rate in treated rotator cuffs is as high as 94%. [See, Li et al. (2023). Nanofibrous scaffolds for the healing of the fibrocartilaginous enthesis: advances and prospects. Nanoscale Horizons, 8(10), 1313-1332. https://doi.org/10.1039/d3nh00212h]

Currently, the gold standard of care for rotator cuff tears involves arthroscopic single or double-row repair in which one or two rows, respectively, of suture anchors are implanted into the humeral head. These anchors are used to suture the torn tendon onto the bone or reattach the torn tendon onto the humeral head. While arthroscopic rotator cuff repair (ARCR) is a common procedure used to reattach the tendon to bone, the 94% re-tear rate insists that regenerative tendon-strengthening alternatives are imperative to reliable, long-term healing.

A cause of the high re-tear rates in ARCR is due to the formation of reactive scar tissue at the tendon-bone interface. This scar tissue prevents the regeneration of the natural enthesis between the tendon and bone, a tissue gradient comprised of the tendon, bone, and fibrocartilage (nonmineralized and mineralized) layers in between. [See, Hall (2015). Tendon Skeletogenesis and Sesamoids. Bones and Cartilage Developmental and Evolutionary Skeletal Biology, 137-149. https://doi.org/10.1016/b978-0-12-416678-3.00009-4] When fibrous scar tissue takes the place of the natural enthesis, the alignment of the tissue between tendon fibers and bone is disorganized, which leads to a decrease in the mechanical strength of the repaired interface. Thus, the difference in tissue structure and the loss of normal biomechanics when to the native interface leads to the high prevalence of tendon re-tearing.⁵

Many efforts have been made to improve the mechanical stability of the tendon-bone interface by thickening the torn tendon after ARCR procedures. On-lay scaffolds have been placed on top of the tendon to support it mechanically before being sutured onto the bone. One of these products is Smith & Nephew's REGENTEN patch, a porous bovine Achilles tendon-derived implant used for on-lay applications. [See, Rognoni et al. (2023). Economic Evaluation of a Bioinductive Implant for the Repair of Rotator Cuff Tears Compared with Standard Surgery in Italy. Advances in Therapy, 40(12), 5271-5284. https://doi.org/10.1007/s12325-023-02686-9] In-lay scaffolds, or interpositional scaffolds, are placed between the tendon and bone to help proliferate tenocytes, chondrocytes, and osteocytes as it also supports the tendon mechanically.

Zimmer Biomet's BioWick is also available as an interpositional scaffold composed of aligned electrospun PLGA nanofibers. [See, Easley et al. (2020). A prospective study comparing tendon-to-bone interface healing using an interpositional bioresorbable scaffold with a vented anchor for primary rotator cuff repair in sheep. Journal of Shoulder and Elbow Surgery, 29(1), 157-166. https://doi.org/10.1016/j.jse.2019.05.024] These products help thicken the tendon; however, due to their fast degradation rates and inability to regenerate anatomical enthesis organization, these products do not improve long-term healing and address the presence of fibrous tissue formation in patients. [See, Chainani et al. (2016). Current Status of Tissue-engineered Scaffolds for Rotator Cuff Repair. Techniques in Orthopaedics, 31(2), 91-97. https://doi.org/10.1097/bto.0000000000000168]

### SUMMARY

According to the present disclosure, highly advantageous enthesis grafting materials are fabricated from citrate-based materials. The disclosed grafting materials can be used to form biodegradable rotator cuff patches that can regenerate the naturally occurring enthesis layers.

In exemplary embodiments, a patch system is provided that may be used in musculoskeletal repair, e.g., in rotator cuff repair. The patch system is generally fabricated, in whole or in part, from a composition that includes a citrate polymer and a bioceramic filler. The disclosed patch system defines a patch thickness characterized by a porosity gradient across the patch thickness. The patch system may include a patch that is defined by a plurality of patch layers. The patch layers may be formed from a plurality of fibers. First fibers in a first patch layer may be non-aligned relative to second fibers in a second, adjacent patch layer. First fibers in a first patch layer may be aligned parallel relative to the tendon fiber upon insertion.

In an embodiment, each of the patch layers may define an aperture, and the apertures of the patch layers of the patch layers may be substantially aligned when the patch is assembled. The patch may define a solid perimeter. The patch may define an open or unbounded perimeter.

The patch system may include a bioceramic filler that is selected from the group consisting of bioactive glasses (BG), hydroxyapatite (HA), tricalcium phosphate (TCP), calcium sulfate, and combinations thereof. The patch may be formed from a plurality of patch layers, and the bioceramic filler level may vary across the patch layers. The patch may be formed from a plurality of patch layers, and the bioceramic filler level may be uniform across the patch layers.

In an embodiment, the patch may define a first patch phase that is configured to be positioned adjacent a bone, and a final patch phase that is configured to be positioned adjacent a tendon. In an embodiment, the bioceramic filler may be at a greater level in the first patch phase as compared to the final patch phase.

In an embodiment, the patch may define a first patch phase that is configured to be positioned adjacent a bone, and a final patch phase that is configured to be positioned adjacent a tendon. In an embodiment, the bioceramic filler may be uniform across all patch phases.

In an embodiment, the patch may define a first patch phase that is configured to be positioned adjacent a bone, and a final patch phase that is configured to be positioned adjacent a tendon. In an embodiment, the porosity may be greater for the first patch phase as compared to the final patch phase. In an embodiment, the patch may define a first patch phase that is configured to be positioned adjacent a bone, and a final patch phase that is configured to be positioned adjacent a tendon, and the porosity may be uniform across all patch phases. In an embodiment, the pore size of a first patch phase may be 300-500 microns, the pore size of a second patch phase is 200-400 microns, and the pore size of a final patch phase is 100-300 microns. In an embodiment, the pore size of all patch phases may be 300-500 microns.

In an embodiment, the degradation profile of the patch may be under 18 months.

The patch may be fabricated from a plurality of fibers, wherein the diameter of the fibers is 100 micron to 500 microns. The patch thickness may be under 2 mm. The patch dimensions may be on the order of 20 mm×20 mm.

In an embodiment, a patch system for use in musculoskeletal repair, e.g., rotator cuff repair, is provided that includes a patch that defines a patch thickness, the patch fabricated, in whole or in part, from a composition that includes a citrate polymer and a bioceramic filler. The patch may be defined by of a plurality of patch layers, and each of the plurality of patch layers may define an aperture. The apertures of the plurality of patch layers may be substantially aligned when the patch is assembled. Each of the apertures may be bounded.

In an embodiment, a patch system is provided for use in musculoskeletal repair, e.g., rotator cuff repair, that includes a patch that defines a patch thickness, the patch fabricated, in whole or in part, from a composition that includes a citrate polymer and a bioceramic filler. The patch may be defined by of a plurality of patch layers. First fibers in a first patch layer may be non-aligned relative to second fibers in a second, adjacent patch layer. The first fibers in the first patch layer and the second fibers in the second, adjacent patch layer may be in non-parallel alignment relative to each other. The first fibers in the first patch layer and the second fibers in the second, adjacent patch layers may intersect at a 60-degree angle relative to each other.

In exemplary embodiments, the disclosed patch layers may be fabricated from a composition that includes (i) a citrate and/or other carboxylic acid component, (ii) a polyol, and (iii) particulate inorganic material. The citrate component may include one or more of citric acid, citrate, or an ester of citric acid. The carboxylic acid component may include carboxylic anhydrides, such as maleic anhydride. The polyol may include a diol, e.g., one or more of butanediol, hexanediol, octanediol, or polyethylene glycol. Other exemplary polyols contemplated according to the present disclosure include one or more of glycerol, beta-glycerol phosphate, or xylitol.

The disclosed citrate and polyol may be reacted, for example, at a 1.0:1.0 to 1.0:1.5 molar ratio, respectively, to form a telechelomer. The citric acid molar ratio can be substituted at a 1-50 mol % with other carboxylic acids or anhydrides including maleic anhydride to tune the molecular weight, degradation profile, and mechanical properties of the polymer. In exemplary embodiments, the polyol may include glycerol at 1-40 mol % of the total polyol included in the composition. In other exemplary embodiments, the polyol may include beta-glycerol phosphate at 1-100 mol %, and preferably 1-40 mol %, of the total polyol included in the composition. The polyol may include xylitol at 1-100 mol % and, preferably 1-40 mol %, of the total polyol included in the composition.

The disclosed particulate inorganic material may include one or more of hydroxyapatite, tricalcium phosphate, biphasic calcium phosphate, calcium carbonate, carbonated apatite, and Bioactive glass (Bioglass). The particulate inorganic material may also be coated with Bioglass. The particulate inorganic material may include a bioceramic present between 0 and 60 wt.-% of the composition. The particulate inorganic material may include a bioceramic that is micro-sized or nano-sized and/or rod-shaped bioceramic.

In exemplary embodiments, a patch system may be formed at least in part from the disclosed composition. The patch may be or define a crosslinked polymer network and is biodegradable. The patch system may be >50% porous and conformable for arthroscopic procedural requirements. The patch may have varying strut sizes and pore sizes to accommodate the infiltration of various cell types within the enthesis. The patch may fully degrade in a time period between 4-12 months in vivo.

Additional features, functions and benefits of the disclosed scaffold will be apparent from the description which follows, particularly when read in conjunction with the accompanying figures.

### BRIEF DESCRIPTION OF THE FIGURES

To assist those of skill in the art in making and using the disclosed patch systems, reference is made to the accompanying figures, wherein:

FIG. 1A is a schematic depiction of a multi-phase scaffold/patch system with gradient pore and strut sizes across the patch layers;

FIG. 1B is a schematic depiction of a multi-phase scaffold/patch system with uniform pore and strut sizes across the patch layers;

FIG. 1C is a schematic depiction of a multi-phase scaffold/patch system with gradient pore and strut sizes across the patch layers that includes a passage extending through the patch layers;

FIG. 1D is a schematic depiction of a multi-phase scaffold/patch system with uniform pore and strut sizes across the patch layers that includes a passage extending through the patch layers;

FIGS. 2A-2C are photographs of each phase of an implementation of the gradient lattice design of FIG. 1A;

FIG. 3 is a SEM image of a citrate-based patch (POMaC composited with 30% HA and 30% Bioglass) showing a gradient pore and strut size design according to the present disclosure;

FIG. 4A is a Brightfield microscopy image of a citrate-based patch (POMaC composited with 30% HA and 30% Bioglass) showing the aligned tendon phase having a uniform pore and strut size across the patch layers; and

FIG. 4B is a Brightfield microscopy image of a citrate-based patch (POMaC composited with 30% HA and 30% Bioglass) showing the bone adjacent phase having a uniform pore and strut size across the patch layers.

### DESCRIPTION OF EXEMPLARY EMBODIMENTS

To overcome the limitations of current scaffolds fabricated from electrospun thermoplastic polymers, citrate-based biodegradable elastomer bioceramic composites have been developed as synthetic bioenergetic scaffolds for enthesis regeneration. Because citric acid is an inexpensive reagent, biocompatible, and plays a critical role in the metabolic cycle within the body, there are many benefits to incorporating it into a biodegradable polymer. For example, citric acid aids in maintaining bone morphology and physiology by regulating apatite development and energy production. [See, Tran et al. (2015). Citrate-Based Biomaterials and Their Applications in Regenerative Engineering. Annual review of materials research, 45, 277-310. https://doi.org/10.1146/annurev-matsci-070214-0208153] When incorporated into polymer chains, the pendant carboxylic acid and hydroxyl groups from citric acid can also function for crosslinking and for improved bioceramic interactions.

To be effective, synthetic enthesis grafts should be able to regenerate all four layers of tissue that connect tendon to bone: tendon, nonmineralized fibrocartilage, mineralized fibrocartilage, and bone. Each layer requires a unique architecture providing the correct pore size, fiber size, and texture to allow each cell type to effectively infiltrate and proliferate into the scaffold with the correct orientation.

Because tendon fibers are highly organized into bundles of aligned collagenic fibers and fibroblasts, the present disclosure provides a patch system that mimics the native anatomy of the tendon by incorporating struts, e.g., 3D-printed struts, that align with the parallel direction of the tendon fibers to promote directional growth of the tenocytes within the tendon-adjacent layer. Directional tendon growth improves the mechanical strength of the tendon and allows for a more efficient transfer of loads from the muscle.

While 3D printing can allow extrusion and control over the fiber size, there are many other ways that the disclosed patch could be fabricated, in whole or in part, to effectively provide variability in the size and/or direction of the fibers. Such methods include casting each layer separately and then combining the layers. A further modality for patch fabrication involves a sacrificial mold that would allow the citrate-based biodegradable elastomer bioceramic composites to be poured into the mold, solidified and cured, and then the mold could be dissolved using an appropriate substance/solvent/energy source. Alternatively, the disclosed patch could be fabricated from solid planks that have alternating grooves machined into them, thereby creating a pair of layers that could then be stacked with other layer pairs to create a variable pore structure, as desired. Thus, a range of fabrication modalities may be employed to form the disclosed scaffolds/path systems, that include molding (e.g., casting), additive manufacturing (e.g., 3D Printing), and/or subtractive manufacturing (e.g., machining) to create the desired variable filament/pore size.

In an embodiment, a homogenous design may be provided that incorporates layers with a 400 μm pore size and a 400 μm strut diameter. In a different configuration, the patch design can be divided into phases that more closely address the architecture requirements of each layer. The tendon phase may have a pore and strut size of 150 μm, the cartilage phase may have a pore and strut size of 250 μm, and the bone phase may have a pore and strut size of 400 μm. In doing so, each cell type's infiltration and attachment may be more adequate within the patch layers.

One way to tune the fiber size, direction, and spacing is through 3D printing the porous structure, which allows for precise control over each layer's attributes. This will effectively create a gradient structure that can stimulate the enthesis tissues' growth, proliferation, and maturation throughout.

As noted above, patch systems according to the present disclosure are fabricated from citrate-based materials. The disclosed materials may be used to fabricate a biodegradable patch. In exemplary embodiments, the disclosed materials include a composition that includes (i) a citrate component and/or a carboxylic anhydride component, (ii) a polyol, and (iii) particulate inorganic material. Additional details of exemplary implementations of the disclosed materials and patch systems are described herein with reference to the accompanying figures.

In an exemplary implementation, a citrate-based scaffold/patch system is provided that includes a 20×20 mm patch with a 1-2 mm thickness. These dimensions were chosen for illustrative purposes to balance structural integrity with flexibility for enthesis repair and regeneration. A thickness range from 1 to 2 mm allows the scaffold to be robust enough to be handled during implantation yet thin enough to be pliable. Although the noted dimensions offer a clinically beneficial design, the present disclosure is not limited by or to such exemplary dimensions.

3D printing allows for design flexibility in scaffold/patch system fabrication. For example, 3D printing supports the fabrication of patches/patch layers that feature (i) uniform, consistent, and/or homogenous pore size, and/or (ii) gradient pore size distribution throughout the scaffold/patch system, e.g., as formed from an assembly of patch layers. For a uniform scaffold/patch system design, each phase may contain fibers and pores of a consistent size throughout the scaffold/patch system. The uniformly sized pores may be beneficial for better diffusion throughout the scaffold/patch system, allowing for greater cell migration from different cell types.

A gradient scaffold incorporates multiple phases with variability between at least two of the phases. For example, each phase may be characterized by its own pore size and fiber diameter, and the pore size and/or fiber diameter may vary across each (or, for example, several) of the phases. In an embodiment, a gradient scaffold design may be provided, wherein the phase to be positioned adjacent to the tendon has the tightest pore size within the scaffold/patch system. In embodiments where the fibers are 3D printed, the fibers printed in the layer to be positioned adjacent the tendon may be oriented so as to be parallel to the natural striations of the tendon. The fiber alignment in this layer may strongly influence and benefit the proliferation of tenocytes. [See, Su et al.] In contrast, the phase adjacent to the bone may have the largest pore size within the scaffold/patch system. According to the present disclosure, a gradient in pore sizes and fiber diameters can be fabricated to tailor the mechanical properties to different regions and promote multi-tissue regeneration.

The 3D-printed layers may be designed so that overlapping layers form a lattice structure. In an embodiment, a first layer may be designed for the tendinous phase with orthogonally printed fibers (to be aligned with tendon fiber growth), while the adjacent layer fibers may be printed with an angular orientation, e.g., a 30-degree rotation, relative thereto. Subsequently, each scaffold layer may be designed with an angular orientation, e.g., a 60-degree rotation, relative to the adjacent layer, forming a non-isotropic lattice structure. The foregoing angular orientations may be varied without departing from the present disclosure. For example, the disclosed 30-degree rotation could range from about 35-degree to 45-degree rotation. Similarly, the disclosed 60-degree rotation could range from about 55-degree to 65-degree rotation.

In an embodiment, the non-isotropic angular offset contributes to forming a strong, interlocking matrix that enhances the overall mechanics and flexibility of the scaffold/patch system. The lattice configuration offers increased structural stability and uniform stress distribution, while facilitating easier handling and rolling of the scaffold to meet the requirements of arthroscopic procedures. [See, Montgomery et al. (2017). Flexible shape-memory scaffold for minimally invasive delivery of functional tissues. Nature Materials, 16(10), 1038-1046. https://doi.org/10.1038/nmat4956]

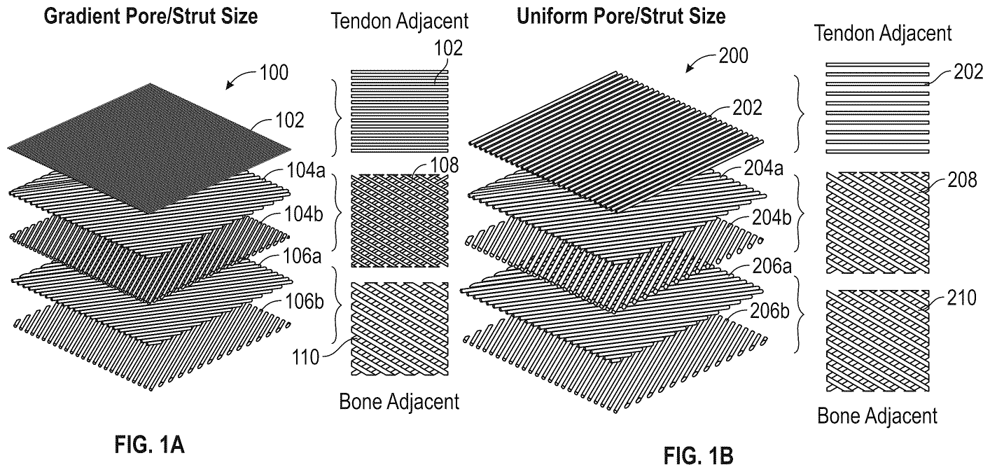

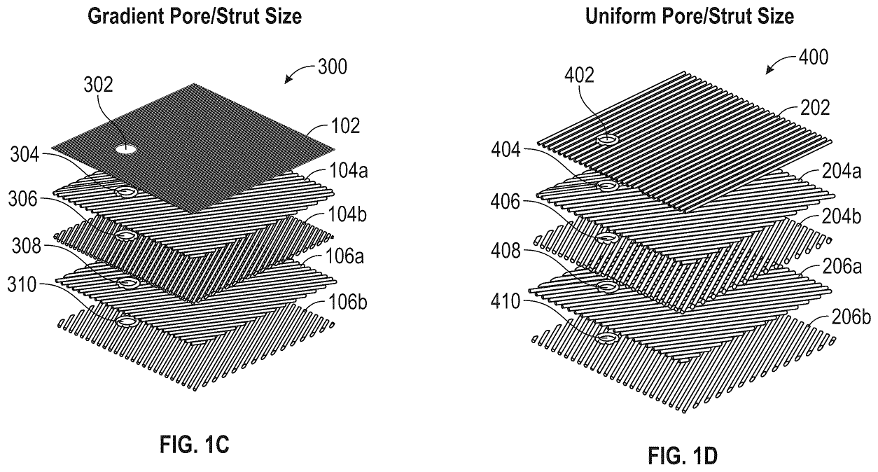

With reference to FIG. 1A, a schematic depiction of a multi-phase scaffold/patch system 100 with gradient lattice design across the patch layers according to the present disclosure is provided. Although depicted in an exploded manner, the adjacent layers are in an abutting/contacting relation. For illustration purposes, five (5) layers are schematically depicted: a tendon-adjacent layer 102, a pair of layers 104a, 104b that are angularly oriented relative to each other, and a second pair of layers 106a, 106b that are angularly oriented relative to each other. Layer 106b is bone-adjacent in the schematically depicted embodiment. Angular orientation of the fibers from layer-to-layer is apparent as schematically depicted, for example, by paired layers 104a/104b, shown as combination 108, and paired layers 106a/106b, shown as combination 110.

With reference to FIG. 1B, a schematic depiction of a multi-phase scaffold/patch system with uniform lattice design across the patch layers according to the present disclosure is provided. As with the schematic depiction of the gradient lattice design of FIG. 1A, although depicted in an exploded manner, the adjacent layers are in an abutting/contacting relation. Angular orientation of the fibers from layer-to-layer and alignment of the fibers relative of the “tendon adjacent” layer is shown. Specifically, for illustration purposes, five (5) layers are schematically depicted: a tendon-adjacent layer 202, a pair of layers 204a, 204b that are angularly oriented relative to each other, and a second pair of layers 206a, 206b that are angularly oriented relative to each other. Layer 206b is bone-adjacent in the schematically depicted embodiment. Angular orientation of the fibers from layer-to-layer is apparent as schematically depicted, for example, by paired layers 204a/204b, shown as combination 208, and paired layers 206a/206b, shown as combination 210

With reference to FIGS. 1C and 1D, schematic depictions of multi-phase scaffold/patch systems 300 and 400 are provided. Patch system 300 corresponds to patch system 100, except that at least one aperture/passage is defined in the face of each patch layer (apertures 302, 304, 306, 308, 310) and, when assembled, the apertures/passages 302-310 are substantially aligned to define a contiguous passage through patch system 300. The disclosed contiguous passage provides room/space for formation of natural host tissues (i.e., bone-enthesis-tendon). Similarly, patch system 400 corresponds to patch system 200, except that at least one aperture/passage is defined in the face of each patch layer (apertures 402, 404, 406, 408, 410) and, when assembled, the apertures/passages 402-410 are substantially aligned to define a contiguous passage through patch system 400. The disclosed contiguous passage provides room/space for formation of natural host tissues (i.e., bone-enthesis-tendon).

To demonstrate the strut and pore architecture of a biodegradable patch fabricated according to the present disclosure, citrate-based polymers (e.g., poly (octamethylene co-maleate citrate) (POMaC)), were composited with 30 wt.-% hydroxyapatite (HA) and 30 wt.-% 45S5 Bioactive Glass (Bioglass) (POMaC H3B3).

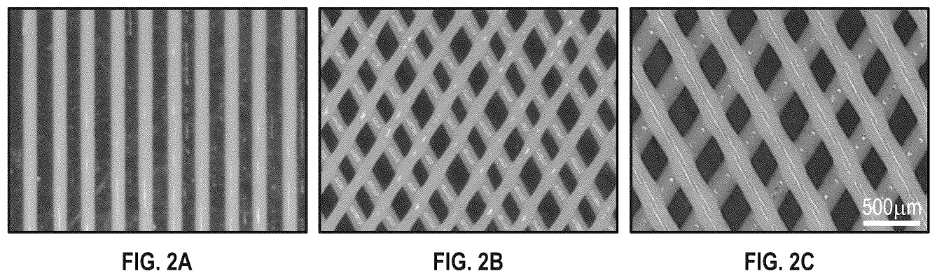

FIGS. 2A-2C provide photographic views of each phase of the gradient patch of FIG. 1A. Specifically, FIGS. 2A-2C are Brightfield microscopy images (top views) of a citrate-based patch (POMaC composited with 30% HA and 30% Bioglass). FIG. 2A shows a phase that, when implanted, will align with a tendon. FIG. 2B shows a phase that, when implanted, will align with cartilage. FIG. 2C shows a phase that, when implanted, will align with bone. The foregoing phases may be printed sequentially using 3D printing technology, and then combined to form an interpositional patch with a pore and strut size gradient throughout the device.

In the microscopy image of FIG. 2A, the tendon-adjacent layer has a 150 μm pore diameter and a 350 μm strut diameter. In the microscopy image of FIG. 2B, the middle phase of the scaffold/patch system has layers with a 250 μm pore diameter and a 250 μm strut diameter optimized for cartilage regeneration. In the microscopy image of FIG. 2C, within the phase configured to be adjacent to bone, the layers have a 500 μm pore diameter and a 350 μm strut diameter. The foregoing dimensional parameters may be varied without departing from the present disclosure.

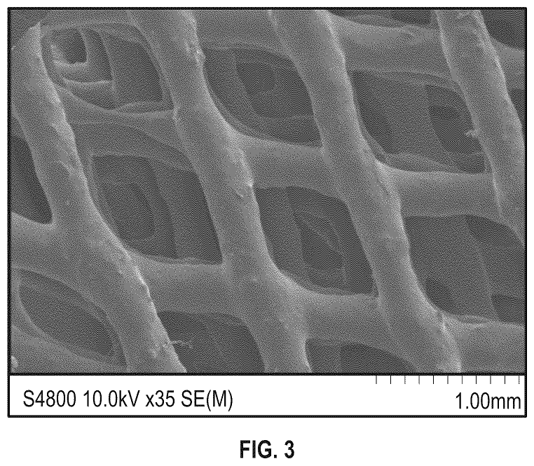

With reference to FIG. 3, an SEM image is provided showing the gradient pore and strut size of a citrate-based patch (POMaC composited with 30% HA and 30% Bioglass) according to the present disclosure. The individual layers may be 3D printed sequentially and then assembled, thereby forming a scaffold/patch system in which the combination of phases forms a gradient pore and strut size scaffold.

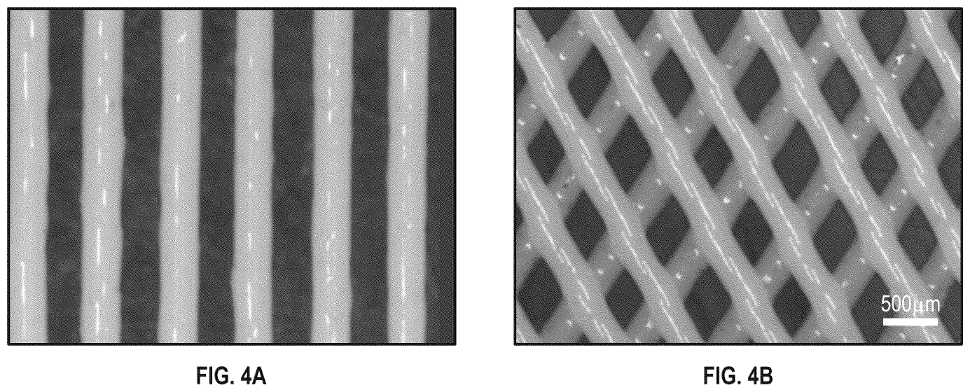

With reference to FIGS. 4A and 4B, Brightfield microscopy images of a citrate-based homogeneous patch (POMaC composited with 30% HA and 30% Bioglass) is provided. More specifically, is a Brightfield microscopy image of a citrate-based patch (POMaC composited with 30% HA and 30% Bioglass) showing the aligned tendon phase having a uniform pore and strut size across the patch layers. FIG. 4B is a Brightfield microscopy image of a citrate-based patch (POMaC composited with 30% HA and 30% Bioglass) showing the bone adjacent phase having a uniform pore and strut size across the patch layers.

The homogenous patch layers show a uniform pore and strut diameter within all patch layers. In the exemplary embodiment, these layers have a uniform strut size of 450 μm and a uniform pore size of 450 μm.

1. A patch system for use in musculoskeletal repair, comprising:
a patch that defines a patch thickness, the patch fabricated, in whole or in part, from a composition that includes a citrate polymer and a bioceramic filler; wherein the patch defines a porosity gradient across the patch thickness.

2. The patch system according to claim 1, wherein the patch is defined by a plurality of patch layers.

3. The patch system according to claim 2, wherein the patch layers are formed from a plurality of fibers.

4. The patch system according to claim 3, wherein first fibers in a first patch layer are non-aligned relative to second fibers in a second, adjacent patch layer.

5. The patch system according to claim 3, wherein first fibers in a first patch layer are parallelly aligned relative to the tendon fiber upon insertion.

6. The patch system according to claim 1, wherein the patch defines a solid perimeter.

7. The patch system according to claim 1, wherein the patch defines an open or unbounded perimeter.

8. The patch system according to claim 1, wherein the bioceramic filler is selected from the group consisting of bioactive glasses (BG), hydroxyapatite (HA), tricalcium phosphate (TCP), calcium sulfate, and combinations thereof.

9. The patch system according to claim 1, wherein the patch is formed from a plurality of patch layers, and wherein the bioceramic filler level varies across the patch layers.

10. The patch system according to claim 1, wherein the patch is formed from a plurality of patch layers, and wherein the bioceramic filler level is uniform across the patch layers.

11. The patch system according to claim 1, wherein the patch defines a first patch phase that is configured to be positioned adjacent a bone, and a final patch phase that is configured to be positioned adjacent a tendon, and wherein the bioceramic filler is at a greater level in the first patch phase as compared to the final patch phase.

12. The patch system according to claim 1, wherein the patch defines a first patch phase that is configured to be positioned adjacent a bone, and a final patch phase that is configured to be positioned adjacent a tendon, and wherein the bioceramic filler is uniform across the first and the final patch phases.

13. The patch system according to claim 1, wherein the patch defines a first patch phase that is configured to be positioned adjacent a bone, and a final patch phase that is configured to be positioned adjacent a tendon, and wherein the porosity is greater for the first patch phase as compared to the final patch phase.

14. The patch system according to claim 1, wherein the patch defines a first patch phase that is configured to be positioned adjacent a bone, and a final patch phase that is configured to be positioned adjacent a tendon, and wherein the porosity is uniform across the first and the final patch phases.

15. The patch system according to claim 1, wherein the patch defines a first patch phase that is configured to be positioned adjacent a bone, a final patch phase that is configured to be positioned adjacent a tendon, and a second patch phase positioned between the first patch phase and the final patch phase, and wherein the pore size of the first patch phase is 300-500 microns, the pore size of the second patch phase is 200-400 microns, and the pore size of the final patch phase is 100-300 microns.

16. The patch system according to claim 1, wherein the patch defines a first patch phase that is configured to be positioned adjacent a bone, a final patch phase that is configured to be positioned adjacent a tendon, and a second patch phase positioned between the first patch phase and the final patch phase, and wherein the pore size of each of the first, second and final patch phases is 300-500 microns.

17. The patch system according to claim 1, wherein the degradation profile of the patch is under 18 months.

18. The patch system according to claim 1, wherein the patch is fabricated from a plurality of fibers, and wherein the diameter of the fibers is 100 micron to 500 microns.

19. The patch system according to claim 1, wherein the patch thickness is under 2 mm.

20. The patch system according to claim 2, wherein each of the plurality of layers defines an aperture that, when assembled, align to define a contiguous passage through the plurality of layers.

21. A patch system for use in musculoskeletal repair, comprising:
a patch that defines a patch thickness, the patch fabricated, in whole or in part, from a composition that includes a citrate polymer and a bioceramic filler;
wherein the patch is defined by of a plurality of patch layers, and
wherein first fibers in a first patch layer are non-aligned relative to second fibers in a second, adjacent patch layer.

22. The patch system of claim 21, wherein the first fibers in the first patch layer and the second fibers in the second, adjacent patch layer are in non-parallel alignment relative to each other.

23. The patch system of claim 21, wherein the first fibers in the first patch layer and the second fibers in the second, adjacent patch layers intersect at a 60-degree angle relative to each other.
